(19) United States
(12) Patent Application Publication
(10) Pub. No.: US 2024/0410801 A1
(43) Pub. Date: Dec. 12, 2024

(54) METHOD FOR ANALYZING CONTOUR OF ROUND BAR SPECIMEN DURING UNIAXIAL TENSILE NECKING DEFORMATION

(71) Applicant: Luoyang Ship Material Research Institute, Luoyang (CN)

(72) Inventors: Gang XUE, Luoyang (CN); Wei TANG, Luoyang (CN); Xinyao ZHANG, Luoyang (CN)

(21) Appl. No.: 18/737,422

(22) Filed: Jun. 7, 2024

(30) Foreign Application Priority Data
Jun. 9, 2023 (CN) 202310682941.7

(51) Int. Cl. G01N 3/08; G01N 33/20

(52) U.S. Cl. CPC G01N 3/08; G01N 33/20

(57) ABSTRACT

Analyzing a contour of a round bar specimen during uniaxial tensile necking deformation by analyzing a shape of a contour line of a specimen during a necking stage; setting a contour of the specimen at a necking bottom during the necking stage to be S-shaped, and establishing a mathematical model for the contour line; measuring test data of the specimen that has undergone necking deformation, and substituting the measured data into the model to determine shape characteristic values; and substituting the determined values into the model to obtain a curve model of a rotational generatrix of the contour. The method accurately describes the contour rotational generatrix and contour curved surface of the specimen shape during the necking deformation stage in a uniaxial tensile test of a round bar specimen by establishing mathematical models. Advantages include clear physical mechanism, concise mathematical model, and high analytical precision.

### CROSS-REFERENCE TO RELATED APPLICATIONS

This application claims priority to Chinese Application No. 202310682941.7 filed on Jun. 9, 2023, the contents of which are incorporated fully herein by reference.

### TECHNICAL FIELD

The present invention relates to the technical field of material tests, and in particular, relates to a method for analyzing a contour of a round bar specimen during uniaxial tensile necking deformation.

### BACKGROUND

The uniaxial tensile test of round bar specimens is one of the most fundamental methods for testing the mechanical properties of materials. It can measure mechanical property parameters such as yield strength, tensile strength, reduction of area, and elongation after fracture of metal materials. Necking deformation is a common phenomenon in the uniaxial tensile test of round bar specimens for metal materials like low-alloy steel. This phenomenon is characterized by the specimen transitioning into a state of plastic deformation concentrated in part of the region after undergoing a certain degree of uniform plastic deformation. Necking deformation causes the stress state in the centralized deformation region to change from uniaxial stress before necking to triaxial stress after necking. Since the triaxial stress state is related to the specimen shape during necking, establishing a mathematical model of the necking shape is the foundational basis for analyzing the stress field distribution within the necking region.

Chinese Patent CN109883824A discloses a method for inversely calculating the necking arc radius and the minimum necking cross-sectional radius at each moment by measuring the gauge length elongation and the coordinate information of the outer contour curve of the broken specimen during the tensile process of a round bar specimen. Chinese Patent CN113281171A proposes a method for measuring the curvature radius and the minimum cross-sectional diameter of the necking area by real-time acquisition of the necking area contour image and utilizing an optical microscopic measurement system. The above two patents adopt approximate calculation methods of hyperbolic functions or circular arc functions. However, hyperbolic functions or circular arc functions can only approximately describe the shape of the necking bottom and nearby areas of the necking and cannot be used to describe the overall shape of the specimen after necking.

Chinese Patent CN108982222A and Chinese Patent CN108982223A respectively propose methods for metal plate specimens and round bar specimens. The methods involve measuring the instantaneous gauge length and the minimum cross-sectional radius at the necking point after the tensile instability of the specimen, utilizing a necking step model to calculate the necking step coordinates at each moment and then approximating the necking contour curve through the interpolation method, and utilizing the curvature formula to calculate the curvature radius of the minimum cross-section at the necking point at that moment. However, the hypothetical premise for the calculation method is that during the necking deformation process, the deformation is concentrated only at the minimum cross-section, and the regions outside the minimum cross-section do not participate in the deformation. This assumption does not align with the actual situation.

Chinese Patent CN114923774A proposes a mathematical function

$${y}_{neck} = {r}_{s} - ( {r}_{s} - {r}_{n} ) \cdot {[ 1 + \frac{{( x - a )}^{2}}{b} ]}^{- 1}$$

to fit the contour of the necking region (where rₛ represents the radius of the round bar in the non-necking region, rₙ represents the minimum radius in the necking region, α represents the necking position, and b represents the material parameter). For the contour curves of the same specimen at different necking deformation moments,
the radius of the round bar in the non-necking region remains approximately unchanged (the variation is very small and negligible compared to the variation in the minimum cross-sectional radius rₙ of the necking region). Only the minimum radius rₙ of the necking region and the curvature of the contour curve change. In a coordinate system where the center of the minimum cross-section is the origin, the necking position α is zero. Under this condition, when using the aforementioned mathematical function to fit the contour curves at different necking deformation moments, the only parameter to adjust the curvature is the material parameter b. Therefore, the effect of using this mathematical function to fit the necking deformation contour is not ideal. As of now, no mathematical model has been disclosed that can effectively describe the contour of the specimen during the necking deformation stage in uniaxial tensile tests of round bar specimens.

### SUMMARY

In view of this, the present invention aims to provide a method for analyzing the contour line of a round bar specimen during the necking deformation stage in a uniaxial tensile test. By constructing corresponding mathematical models, the objective of characterizing the contour features of the round bar specimen during the necking deformation stage in the uniaxial tensile test is achieved.

The present invention discloses a method for analyzing a contour of a round bar specimen during uniaxial tensile necking deformation. The method analyzes, by conducting a uniaxial tensile test on a round bar specimen, a shape of the specimen during a necking stage in the round bar tensile test, and comprises the following steps:
step S1, analyzing a shape of a contour line of the specimen during the necking stage;
step S2, setting a contour of the specimen at a necking bottom during the necking stage to be S-shaped, setting hypothetical conditions, and establishing a mathematical model for the contour line as shown in equation (1)

$$r = {r}_{n} + \frac{{r}_{c} - {r}_{n}}{1 + {( \frac{z}{{z}_{1}} )}^{{p}_{1}} + {( \frac{z}{{z}_{2}} )}^{{p}_{2}}} \qquad ( 1 )$$

where r represents a cross-sectional radius perpendicular to a central axis at a position of any point on a contour curved surface of the specimen, z represents a distance between a cross-section at the any point and a minimum cross-section at the necking bottom, rₙ represents a maximum limit value of the cross-sectional radius perpendicular to the central axis, r_{c} represents a minimum cross-sectional radius at the necking bottom, and z₁, p₁, z₂, and p₂ represent undetermined shape characteristic parameters;
step S3, measuring test data of the specimen that has undergone necking deformation, wherein the test data comprises at least the cross-sectional radius r and the cross-sectional distance z, and there are a plurality of measurement points; substituting the measured test data into equation (1) for fitting to determine values of rₙ, r_{c}, and the shape characteristic parameters z₁, p₁, z₂, and p₂; and
step S4, substituting the values of rₙ, r_{c}, and the shape characteristic parameters z₁, p₁, z₂, and p₂ determined in step S3 into equation (1) to obtain a curve model of the rotational generatrix.

Further, the hypothetical conditions in step S2 are as follows:
during the necking stage of the uniaxial tensile test of the round bar specimen, a shape of the round bar specimen being a rotational body formed by rotating the rotational generatrix of the contour around the central axis; the specimen being symmetric with respect to the minimum cross-section at the necking bottom along a direction of the central axis; and a tangent line of a contour line at a position of the minimum cross-section is parallel to the central axis.

Further, step S2 further comprises:
step S21, establishing a rectangular coordinate system with a center position of the minimum cross-section at the necking bottom, which is perpendicular to the central axis, as an origin, using the central axis as a z-axis of the coordinate system, and using any two mutually perpendicular radius lines intersecting at the center of the minimum cross-section at the necking bottom as an x-axis and a y-axis of the coordinate system; and
step S22, setting coordinates of any point on the contour curved surface of the specimen as (x, y, z), and according to the mathematical model of the rotational generatrix from step S2, establishing a curved surface model of the contour of the specimen as shown in equation (2)

$$\sqrt{{x}^{2} + {y}^{2}} = {r}_{n} + \frac{{r}_{c} - {r}_{n}}{1 + {( \frac{z}{{z}_{1}} )}^{{p}_{1}} + {( \frac{z}{{z}_{2}} )}^{{p}_{2}}} . \qquad ( 2 )$$

Further, step S4 further comprises:
step S41, substituting the values of rₙ, r_{c}, and the shape characteristic parameters z₁, p₁, z₂, and p₂ determined in step S3 into equation (2) to obtain a curved surface model of the necking deformation contour.

Further, step S2 further comprises:
step S21′, based on the mathematical model of the rotational generatrix in step S2, establishing a mathematical model of a tangent slope at any point on the rotational generatrix of the contour in a plane formed by the rotational generatrix and the central axis, as shown in equation (3)

$${k}_{t} = - ( {r}_{c} - {r}_{n} ) \cdot {[ 1 + {( \frac{z}{{z}_{1}} )}^{p 1} + {( \frac{z}{{z}_{2}} )}^{{p}_{2}} ]}^{- 2} \cdot [ \frac{{p}_{1}}{{z}_{1}^{p 1}} \cdot {z}^{p {1}^{- 1}} + \frac{{p}_{2}}{{z}_{2}^{{p}_{2}}} \cdot {z}^{p {2}^{- 1}} ] \qquad ( 3 )$$

where kₜ represents the tangent slope.

Further, step S4 further comprises:
step S41′, substituting the values of rₙ, r_{c}, and the shape characteristic parameters z₁, p₁, z₂, and p₂ determined in step S3 into equation (3) to obtain the mathematical model of the tangent slope at any point on the rotational generatrix of the contour in the plane formed by the rotational generatrix and the central axis.

Further, after step S41′, the following step is performed:
step S42′, acquiring a tangent slope according to the mathematical model established in step S41′, and determining a maximum value kₜ^{ip} of the tangent slope with a certain precision using a linear search method, the maximum value being a tangent slope at an inflection point position of the rotational generatrix of the contour,
wherein the linear search comprises: setting a series of z values, with a difference between two adjacent z values being a search step size Δz, where Δz represents search precision; calculating tangent slope kₜ corresponding to each z value according to equation (3); and identifying a maximum value of kₜ as kₜ^{p}.

Further, after step S42′, the following step is performed:
step S43′, substituting the z value corresponding to the maximum slope obtained in step S42′ into equation (1) to calculate and acquire a cross-sectional radius rᵢₚ perpendicular to the central axis at the inflection point position.

Further, a number of the measurement points in step S3 is no less than 10.

Further, a material of the round bar specimen is a metal material that undergoes necking deformation during the uniaxial tensile process of the round bar specimen.

Compared to the prior art, the method for analyzing a contour of a round bar specimen during uniaxial tensile necking deformation described in the present invention has the following advantages:

The analysis method provided in the present invention can accurately describe the contour rotational generatrix and contour curved surface of the specimen shape during the necking deformation stage in a uniaxial tensile test of a round bar specimen. The method can also calculate and determine the tangent slope at any point on the contour rotational generatrix in the plane formed by the rotational generatrix and the central axis, as well as calculate and determine the tangent slope at the inflection point of the contour rotational generatrix, the radius of the cross-section perpendicular to the central axis at the inflection point position, and the distance between this cross-section and the minimum cross-section at the necking bottom, among other characteristic parameters that reflect the specimen shape during the necking stage. The analysis method provided in the present invention has the advantages of a clear physical mechanism, a concise mathematical model, and high analytical precision.

### BRIEF DESCRIPTION OF THE DRAWINGS

In order to more clearly illustrate the technical solutions in the embodiments of the present invention or in the prior art, the drawings required to be used in the description of the embodiments or the prior art are briefly introduced below. It is obvious that the drawings in the description below are merely some embodiments of the present invention, and those of ordinary skills in the art can obtain other drawings according to these drawings without creative efforts.

FIG. 1 is a schematic diagram of the specimen shape of a round bar specimen during the necking deformation stage in a uniaxial tensile test according to an embodiment of the present invention;

FIG. 2 is a schematic diagram illustrating the construction of the specimen shape and the rectangular coordinate system of a round bar specimen during the uniaxial tensile necking deformation stage according to an embodiment of the present invention; and

FIG. 3 is a schematic diagram of the specimen shape characteristic parameters during the necking deformation stage according to an embodiment of the present invention.

### DETAILED DESCRIPTION

To make the technical means of the present invention and its objectives and effects easy to understand, the following detailed description of the embodiments of the present invention is provided in conjunction with specific illustrations.

It should be noted that all terms indicating direction and position in the present invention, such as “up”, “down”, “left”, “right”, “front”, “back”, “vertical”, “horizontal”, “inner”, “outer”, “top”, “bottom”, “transverse”, “longitudinal”, and “center”, are used solely to explain the relative positional relationships and connection situations between the components in a specific state (as shown in the drawings). These terms are intended merely for the convenience of describing the present invention and do not necessitate that the present invention be constructed or operated in a specific orientation. Therefore, these terms should not be construed as limiting the present invention. In addition, in the present invention, descriptions involving “first”, “second”, etc., are used for the description purpose only and should not be understood as indicating or implying relative importance or implicitly specifying the quantity of the indicated technical features.

In the description of the present invention, unless otherwise explicitly specified and defined, the terms “install”, “connect”, and “link” should be understood in a broad sense. For example, they may refer to fixed connections, detachable connections, or integral connections; they may be mechanical connections; they may be directly connected or indirectly connected through an intermediary medium, and they may refer to the internal communication between two elements. For those of ordinary skill in the art, the specific meanings of the aforementioned terms in the present invention can be understood according to specific conditions.

In the specification, the reference term “an embodiment”, “some embodiments”, “illustrative embodiments”, “an example”, “a specific example”, or “some examples” means that a specific feature, structure, material, or characteristic described in connection with the embodiment or example is included in at least one embodiment or example of the present invention. In the specification, the schematic description of the aforementioned terms does not necessarily refer to the same embodiment or example. Moreover, the specific feature, structure, material, or characteristic described may be combined in a suitable manner in any one or more embodiments or examples.

The present invention discloses a method for analyzing a contour of a round bar specimen during uniaxial tensile necking deformation. The method analyzes, by conducting a uniaxial tensile test on a round bar specimen, the specimen shape during the necking stage in the round bar tensile test, and comprises the following steps:
step S1, analyzing the shape of the contour line of a specimen during the necking stage;
step S2, setting hypothetical conditions, setting the rotational generatrix of the contour of the specimen at the necking bottom during the necking stage to be S-shaped, and establishing a mathematical model for the rotational generatrix as shown in equation (1)

$$r = {r}_{n} + \frac{{r}_{c} - {r}_{n}}{1 + {( \frac{z}{{z}_{1}} )}^{{p}_{1}} + {( \frac{z}{{z}_{2}} )}^{{p}_{2}}} \qquad ( 1 )$$

where r represents the cross-sectional radius perpendicular to the central axis at the position of any point on the contour curved surface of the specimen, z represents the distance between the cross-section at the any point and the minimum cross-section at the necking bottom, rₙ represents the maximum limit value of the cross-sectional radius perpendicular to the central axis, r_{c} represents the minimum cross-sectional radius at the necking bottom, and z₁, p₁, z₂, and p₂ represent shape characteristic parameters;
step S3, measuring the test data of the specimen that has undergone necking deformation, wherein the test data comprises at least the cross-sectional radius r and the cross-sectional distance z, and there are a plurality of measurement points; substituting the measured test data into equation (1) for fitting to determine the values of rₙ, r_{c}, and the shape characteristic parameters z₁, p₁, z₂, and p₂; and
step S4, substituting the values of rₙ, r_{c}, and the shape characteristic parameters z₁, p₁, z₂, and p₂ determined in step S3 into equation (1) to obtain the curve model of the rotational generatrix.

The mathematical model established in step S2 is a curve model that can describe the contour of each rotational generatrix. The curved surface formed by rotating the generatrix around the central axis can characterize the contour of the necking deformation on one side of the specimen's minimum cross-section, which therefore is used to characterize the contour of the necking deformation. In this embodiment, equation (1) uses a plurality of characteristic parameters rₙ, r_{c}, z₁, p₁, z₂, and p₂ to enable the curve expressed by equation (1) to have a higher degree of freedom, thereby allowing a more precise description of the contour of the necking deformation. It should be understood that during the necking deformation process, the necking portion of the tensile specimen gradually deforms and elongates. During this process, multiple parameters of the specimen will change. In this embodiment, the analysis of the contour using multiple parameters enables the fitted curve to have a higher degree of freedom and more approximate to the changing situation of the specimen contour during the tensile necking stage, significantly improving the precision of the necking deformation contour analysis.

As one example, the hypothetical conditions in step S1 are as follows:

During the necking stage of the uniaxial tensile test of the round bar specimen, the shape of the round bar specimen is a rotational body formed by rotating the rotational generatrix of the contour around the central axis. The specimen is symmetric with respect to the minimum cross-section at the necking bottom along the direction of the central axis. The tangent line of the contour line at the position of the minimum cross-section is parallel to the central axis.

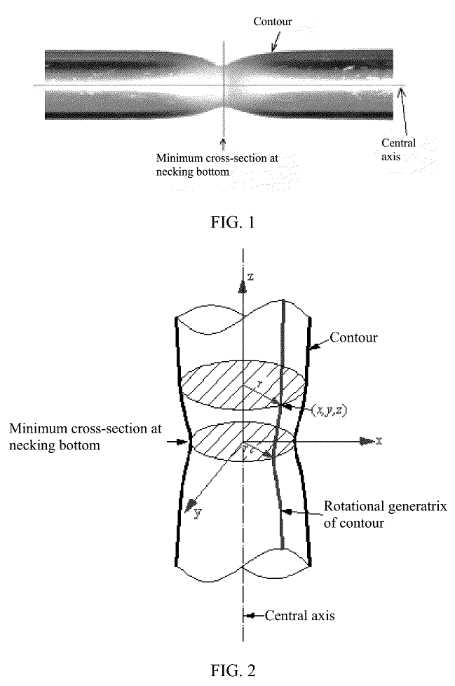

As shown in FIG. 1, the shape of the round bar specimen during the necking stage of a uniaxial tensile test is approximately a rotational body formed by rotating the rotational generatrix of the contour around the central axis. The rotational generatrix is the contour line shown in FIG. 1, and the specimen is symmetric with respect to the minimum cross-section at the necking bottom along the direction of the central axis. The coordinate system established according to step S1 is shown in FIG. 2. The contour line on one side of the cross-section exhibits an “S” shape. At the position of the minimum cross-section, the tangent line of the contour line is parallel to the central axis. The intersection position of the minimum cross-section and the central axis is set as the origin position. The distance between the direction along the central axis and the minimum cross-section is denoted as z. The distribution function of any cross-sectional radius r perpendicular to the central axis with respect to the corresponding cross-section position z is the contour line function of the necking specimen, which is also the rotational generatrix function of the free surface. The contour line during the necking stage can thus be described according to its distribution function.

As another example of the present invention, step S2 further comprises:
step S21, establishing a rectangular coordinate system with the center position of the minimum cross-section at the necking bottom, which is perpendicular to the central axis, as the origin, using the central axis as the z-axis of the coordinate system, and using any two mutually perpendicular radius lines intersecting at the center of the minimum cross-section at the necking bottom as the x-axis and y-axis of the coordinate system; and
step S22, setting the coordinates of any point on the contour curved surface of the specimen as (x, y, z), and according to the mathematical model of the rotational generatrix from step S2, establishing a curved surface model of the contour of the specimen as shown in equation (2)

$$\sqrt{{x}^{2} + {y}^{2}} = {r}_{n} + \frac{{r}_{c} - {r}_{n}}{1 + {( \frac{z}{{z}_{1}} )}^{{p}_{1}} + {( \frac{z}{{z}_{2}} )}^{{p}_{2}}} . \qquad ( 2 )$$

In the rectangular coordinate system established in step S21, the coordinates of any point on the contour curved surface of the specimen have the following relationship with the cross-sectional radius r: x²+y²=r². Combining it with equation (1) can obtain equation (2), which is used to describe the relationship between the coordinates of any point on the contour curved surface of the specimen, thus forming a curved surface function that characterizes the contour of the specimen. Equation (2) is a curved surface function that can directly obtain the overall contour of the specimen during necking deformation, which significantly improves the precision of characterizing the necking deformation contour during the uniaxial tension of a round bar specimen.

Accordingly, step S4 further comprises:
step S41, substituting the values of rₙ, r_{c}, and the shape characteristic parameters z₁, p₁, z₂, and p₂ determined in step S3 into equation (2) to obtain the curved surface model of the necking deformation contour.

As an example of the present invention, step S2 further comprises:
step S21′, based on the mathematical model of the rotational generatrix in step S2, establishing a mathematical model of the tangent slope at any point on the rotational generatrix of the contour in the plane formed by the rotational generatrix and the central axis, as shown in equation (3)

$${k}_{t} = - ( {r}_{c} - {r}_{n} ) \cdot {[ 1 + {( \frac{z}{{z}_{1}} )}^{p 1} + {( \frac{z}{{z}_{2}} )}^{{p}_{2}} ]}^{- 2} \cdot [ \frac{{p}_{1}}{{z}_{1}^{p 1}} \cdot {z}^{p {1}^{- 1}} + \frac{{p}_{2}}{{z}_{2}^{{p}_{2}}} \cdot {z}^{p {2}^{- 1}} ] \qquad ( 3 )$$

where kₜ represents the tangent slope.

In this embodiment, the tangent slope kₜ can help determine the inflection point position of the curve, facilitating further research on the subsequent stress state.

Accordingly, step S4 further comprises:
step S41′, substituting the values of rₙ, r_{c}, and the shape characteristic parameters z₁, p₁, z₂, and p₂ determined in step S3 into equation (3) to obtain the mathematical model of the tangent slope at any point on the rotational generatrix of the contour in the plane formed by the rotational generatrix and the central axis.

As one example, after step S41′, the following step is performed:
step S42′, acquiring a tangent slope according to the mathematical model established in step S41′, and determining the maximum value kₜ^{ip} of the tangent slope with a certain precision using a linear search method, the maximum value being the tangent slope at the inflection point position of the rotational generatrix of the contour.

The linear search comprises: setting a series of z values, with the difference between two adjacent z values being the search step size Δz, where Δz represents the search precision; calculating the tangent slope kₜ corresponding to each z value according to equation (3); and identifying the maximum value of kₜ as kₜ^{ip}.

It should be understood that the tangent slope kₜ calculated based on a series of z values is the tangent slope at a series of equidistant cross-sections spaced by Δz. By using the aforementioned method, the maximum value kₜ^{ip} of the tangent slope with a precision of Δz can be obtained, and the inflection point position is determined based on the maximum value. It should be noted that the linear search may be calculated by using existing general-purpose data processing software, such as Excel, or by writing corresponding calculation programs, which will not be further elaborated or limited here.

For example, by setting the search step size Δz to 0.01 mm, the maximum value of the tangent slope with a precision of 0.01 mm can be obtained, and the subsequent calculation is performed based on this to acquire the required corresponding parameters that reflect the inflection point position of necking deformation, thereby allowing for a more precise description of the contour of the necking deformation. It should be understood that the specific search step size may be set according to requirements and is not limited to the precision provided in the present application.

In one example, after step S42′, the following step is performed:
step S43′, substituting the z value corresponding to the maximum slope obtained in step S42′ into equation (1) to calculate and acquire the cross-sectional radius rᵢₚ perpendicular to the central axis at the inflection point position.

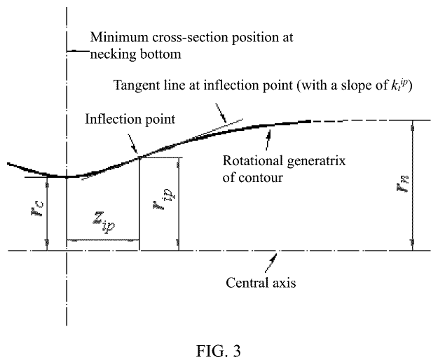

As shown in FIG. 3, determining the parameters at the inflection point position allows for a more precise description of the contour of the necking deformation, which, compared to the prior art, greatly improves the precision of contour analysis and facilitates the subsequent analysis and modeling. Through the above steps, shape characteristic parameters of the round bar specimen can be obtained, such parameters including the minimum cross-sectional radius r_{c}, the maximum limit value rₙ of the cross-sectional radius, the tangent slope kₜ^{ip} at the inflection point, the distance zᵢₚ between the cross-section at the inflection point position and the minimum cross-section (the z value corresponding to the maximum slope), the cross-sectional radius rᵢₚ at the inflection point position, etc. These parameters are conducive to the precise characterization of the necking deformation contour of the specimen and the subsequent modeling and analysis. It should be noted that in the prior art, the maximum limit value rₙ of the cross-sectional radius perpendicular to the central axis cannot be directly measured. It is usually substituted by the measured radius value at the gauge point, but this value is of extremely low accuracy. In the present application, a method for calculating rₙ is proposed, providing a more accurate reference for subsequent research. The tangent slope kₜ^{ip} at the inflection point, the distance zᵢₚ between the cross-section at the inflection point position and the minimum cross-section, and the cross-sectional radius rᵢₚ at the inflection point position are typically analyzed through the method of photography and manual point plotting calculation, which is time-consuming and labor-intensive. Moreover, the limited number of plotted points results in low accuracy of the calculated data. Through the setting of the linear search calculation method in the present application, the above parameters can be obtained directly and quickly, and the calculation precision is much higher than that of the value calculated by manual point plotting in the prior art. By setting up the above technical solution, on one hand, a method for quickly calculating the above shape characteristic parameters is provided, and on the other hand, the precision of the above shape characteristic parameters is significantly improved.

As an example of the present invention, the number of measurement points in step S3 is no less than 10.

As an optional example, the material of the round bar specimen is a metal material that undergoes necking deformation during the uniaxial tensile process of the round bar specimen.

Further, the material of the round bar specimen is one of steel, aluminum alloy, copper alloy, or titanium alloy that undergoes necking deformation during the uniaxial tensile process of the round bar specimen.

Further, the material of the round bar specimen is low-alloy steel that undergoes necking deformation during the uniaxial tensile process of the round bar specimen.

For the specimen shapes of five different round bar specimens during the necking deformation stage in uniaxial tensile tests, the cross-sectional radius r values and the cross-sectional distance z values at 31 points for each specimen are measured. The mathematical model proposed in the present invention is used to fit and determine the specimen shape characteristic parameters r_{c}, rₙ, z₁, p₁, z₂, and p₂. Mathematical models describing the rotational generatrix of the contour, the contour curved surface, and the tangent slope of the rotational generatrix of the contour are established, and the tangent slope at the inflection point of the rotational generatrix of the contour is calculated.

### Embodiment 1

Table 1 shows the measurement data of the cross-sectional radius r and the cross-sectional distance z at 31 points on the contour of the specimen during necking deformation in Embodiment 1.

TABLE 1
Contour Measurement Data of the Specimen in Embodiment 1

| | Measurement Point Number | | | | | | | | | | |
|---|---|---|---|---|---|---|---|---|---|---|---|
| | 1# | 2# | 3# | 4# | 5# | 6# | 7# | 8# | 9# | 10# | 11# |
| Cross-sectional | 0 | 0.50 | 1.00 | 1.50 | 2.00 | 2.50 | 3.00 | 3.50 | 3.99 | 4.49 | 4.99 |
| Distance z (mm) | | | | | | | | | | | |
| Cross-sectional | 3.99 | 4.00 | 4.02 | 4.05 | 4.10 | 4.18 | 4.27 | 4.35 | 4.43 | 4.48 | 4.53 |
| Radius r (mm) | | | | | | | | | | | |
| | Measurement Point Number | | | | | | | | | | |
| | 12# | 13# | 14# | 15# | 16# | 17# | 18# | 19# | 20# | 21# | 22# |
| Cross-sectional | 5.49 | 5.99 | 6.49 | 6.99 | 7.49 | 7.99 | 8.49 | 8.99 | 9.49 | 9.99 | 11.24 |
| Distance z (mm) | | | | | | | | | | | |
| Cross-sectional | 4.56 | 4.59 | 4.65 | 4.66 | 4.69 | 4.7 | 4.72 | 4.73 | 4.74 | 4.74 | 4.75 |
| Radius r (mm) | | | | | | | | | | | |
| | Measurement Point Number | | | | | | | | | | |
| | 23# | 24# | 25# | 26# | 27# | 28# | 29# | 30# | 31# | — | — |
| Cross-sectional | 12.48 | 13.73 | 14.98 | 16.23 | 17.48 | 18.73 | 19.97 | 21.22 | 22.47 | — | — |
| Distance z (mm) | | | | | | | | | | | |
| Cross-sectional | 4.75 | 4.75 | 4.75 | 4.76 | 4.76 | 4.76 | 4.76 | 4.76 | 4.76 | — | — |
| Radius r (mm) | | | | | | | | | | | |

The above data were fitted using the mathematical model of the rotational generatrix of the contour shown in equation (1). The fitted values of the shape characteristic parameters r_{c}, rₙ, z₁, p₁, z₂, and p₂ are shown in Table 2.

TABLE 2
Fitting Results of Embodiment 1

| Parameter | r_{c} | rₙ | z₁ | p₁ | z₂ | p₂ |
|---|---|---|---|---|---|---|
| Fitted Value | 3.99393 | 4.75626 | 6.95496 | 10.74205 | 3.68981 | 2.81136 |
| Standard | 0.00432 | 0.00235 | 0.30851 | 2.08270 | 0.07456 | 0.07456 |
| Error | | | | | | |

The coefficient of determination R² for the fitting is 0.99933, indicating that using the mathematical model shown in equation (1) can effectively describe the rotational generatrix of the contour of the specimen shape. Based on the fitting results, the obtained mathematical models describing the rotational generatrix of the contour of the specimen shape, the contour curved surface, and the tangent slope of the rotational generatrix of the contour in the plane formed by this line and the central axis are given by equations (4), (5), and (6), respectively.

$$r = 4.75626 + \frac{- 0.76233}{1 + {( \frac{z}{6.95496} )}^{10.74205} + {( \frac{z}{3.68981} )}^{2.81136}} \qquad ( 4 )$$

$$\sqrt{{x}^{2} + {y}^{2}} = 4.75626 + \frac{- 0.76233}{1 + {( \frac{z}{6.95496} )}^{10.74205} + {( \frac{z}{3.68981} )}^{2.81136}} \qquad ( 5 )$$

$${k}_{t} = \frac{7.33262 \times 1 {0}^{- 9} \cdot {z}^{9.74205} + 0.05 \; 458 \cdot {z}^{1.81136}}{{[ 1 + {( \frac{z}{6.95496} )}^{10.74205} + {( \frac{z}{3.68981} )}^{2.81136} ]}^{2}} \qquad ( 6 )$$

Table 3 shows the values of the shape characteristic parameters of the specimen's necking deformation contour obtained according to the fitting process of Embodiment 1 and equations (6) and (4).

TABLE 3
Specimen Shape Characteristic Parameters in Embodiment 1

| | | | | Distance zᵢₚ | |
|---|---|---|---|---|---|
| | | | | between | |
| | | Maximum | | Cross-section | Cross- |
| | | Limit | | at | sectional |
| | | Value rₙ | | Inflection | Radius |
| | Minimum | of | Tangent | Point | rᵢₚ at |
| | Cross- | Cross- | Slope | Position and | Inflection |
| | sectional | sectional | kₜ^{ip} at | Minimum | Point |
| Characteristic | Radius r_{c} | Radius | Inflection | Cross-section | Position |
| Parameter | (mm) | (mm) | Point | (mm) | (mm) |
| Value | 3.99393 | 4.75626 | 0.16533 | 2.83463 | 4.23998 |

### Embodiment 2

Table 4 shows the measurement data of the cross-sectional radius r and the cross-sectional distance z at 31 points on the contour of the specimen during necking deformation in Embodiment 2.

TABLE 4
Contour Measurement Data in Embodiment 2

| | Measurement Point Number | | | | | | | | | | |
|---|---|---|---|---|---|---|---|---|---|---|---|
| | 1# | 2# | 3# | 4# | 5# | 6# | 7# | 8# | 9# | 10# | 11# |
| Cross-sectional | 0 | 0.50 | 1.00 | 1.50 | 1.99 | 2.49 | 2.99 | 3.49 | 3.99 | 4.49 | 4.99 |
| Distance z (mm) | | | | | | | | | | | |
| Cross-sectional | 3.80 | 3.81 | 3.84 | 3.89 | 3.95 | 4.04 | 4.14 | 4.19 | 4.29 | 4.36 | 4.43 |
| Radius r (mm) | | | | | | | | | | | |
| | Measurement Point Number | | | | | | | | | | |
| | 12# | 13# | 14# | 15# | 16# | 17# | 18# | 19# | 20# | 21# | 22# |
| Cross-sectional | 5.49 | 5.98 | 6.48 | 6.98 | 7.48 | 7.98 | 8.48 | 8.98 | 9.48 | 9.97 | 11.22 |
| Distance z (mm) | | | | | | | | | | | |
| Cross-sectional | 4.49 | 4.54 | 4.59 | 4.63 | 4.65 | 4.68 | 4.70 | 4.71 | 4.73 | 4.74 | 4.74 |
| Radius r (mm) | | | | | | | | | | | |
| | Measurement Point Number | | | | | | | | | | |
| | 23# | 24# | 25# | 26# | 27# | 28# | 29# | 30# | 31# | — | — |
| Cross-sectional | 12.47 | 13.71 | 14.96 | 16.21 | 17.45 | 18.7 | 19.95 | 21.19 | 22.44 | — | — |
| Distance z (mm) | | | | | | | | | | | |
| Cross-sectional | 4.75 | 4.76 | 4.76 | 4.76 | 4.76 | 4.76 | 4.76 | 4.76 | 4.76 | — | — |
| Radius r (mm) | | | | | | | | | | | |

The above data were fitted using the mathematical model of the rotational generatrix of the contour shown in equation (1). The fitted values of the shape characteristic parameters r_{c}, rₙ, z₁, p₂, z₂, and p₂ are shown in Table 5.

TABLE 5
Fitting Results of Embodiment 2

| Parameter | r_{c} | rₙ | z₁ | p₁ | z₂ | p₂ |
|---|---|---|---|---|---|---|
| Fitted Value | 3.80085 | 4.76075 | 5.98987 | 6.45342 | 4.07690 | 2.27924 |
| Standard Error | 0.00427 | 0.00222 | 0.18635 | 0.54400 | 0.07235 | 0.08822 |

The coefficient of determination R² for the fitting is 0.99970, indicating that using the mathematical model shown in equation (1) can effectively describe the rotational generatrix of the contour of the specimen shape. Based on the fitting results, the obtained mathematical models describing the rotational generatrix of the contour of the specimen shape, the contour curved surface, and the tangent slope of the rotational generatrix of the contour in the plane formed by this line and the central axis are given by equations (7), (8), and (9), respectively.

$$r = 4. 76075 + \frac{- 0.95990}{1 + {( \frac{z}{5.98987} )}^{6.45342} + {( \frac{z}{4.0769} )}^{2.27924}} \qquad ( 7 )$$

$$\sqrt{{x}^{2} + {y}^{2}} = 4.76075 + \frac{- 0.95990}{1 + {( \frac{z}{5.98987} )}^{6.45342} + {( \frac{z}{4.0769} )}^{2.27924}} \qquad ( 8 )$$

$${k}_{t} = \frac{5.95683 \times 1 {0}^{- 5} \cdot {z}^{5.45342} + 0.08 \; 891 \cdot {z}^{1.27924}}{{[ 1 + {( \frac{z}{5.98987} )}^{6.45342} + {( \frac{z}{4.0769} )}^{2.27924} ]}^{2}} \qquad ( 9 )$$

Table 6 shows the values of the shape characteristic parameters of the specimen's necking deformation contour obtained according to the fitting process of Embodiment 2 and equations (9) and (7).

TABLE 6
Specimen Shape Characteristic Parameters in Embodiment 2

| | | | | Distance zᵢₚ | |
|---|---|---|---|---|---|
| | | | | between | |
| | | Maximum | | Cross-section | Cross- |
| | | Limit | | at | sectional |
| | | Value rₙ | | Inflection | Radius |
| | Minimum | of | Tangent | Point | rᵢₚ at |
| | Cross- | Cross- | Slope | Position and | Inflection |
| | sectional | sectional | kₜ^{ip} at | Minimum | Point |
| Characteristic | Radius r_{c} | Radius | Inflection | Cross-section | Position |
| Parameter | (mm) | (mm) | Point | (mm) | (mm) |
| Value | 3.80085 | 4.76075 | 0.16994 | 2.89297 | 4.10627 |

### Embodiment 3

Table 7 shows the measurement data of the cross-sectional radius r and the cross-sectional distance z at 31 points on the contour of the specimen during necking deformation in Embodiment 3.

TABLE 7
Contour Measurement Data in Embodiment 3

| | Measurement Point Number | | | | | | | | | | |
|---|---|---|---|---|---|---|---|---|---|---|---|
| | 1# | 2# | 3# | 4# | 5# | 6# | 7# | 8# | 9# | 10# | 11# |
| Cross-sectional | 0 | 0.50 | 0.99 | 1.49 | 1.99 | 2.49 | 2.98 | 3.48 | 3.98 | 4.48 | 4.97 |
| Distance z (mm) | | | | | | | | | | | |
| Cross-sectional | 3.59 | 3.61 | 3.67 | 3.76 | 3.85 | 3.94 | 4.03 | 4.14 | 4.23 | 4.30 | 4.37 |
| Radius r (mm) | | | | | | | | | | | |
| | Measurement Point Number | | | | | | | | | | |
| | 12# | 13# | 14# | 15# | 16# | 17# | 18# | 19# | 20# | 21# | 22# |
| Cross-sectional | 5.47 | 5.97 | 6.47 | 6.96 | 7.46 | 7.96 | 8.46 | 8.95 | 9.45 | 9.95 | 11.19 |
| Distance z (mm) | | | | | | | | | | | |
| Cross-sectional | 4.44 | 4.49 | 4.53 | 4.58 | 4.60 | 4.63 | 4.66 | 4.69 | 4.70 | 4.71 | 4.73 |
| Radius r (mm) | | | | | | | | | | | |
| | Measurement Point Number | | | | | | | | | | |
| | 23# | 24# | 25# | 26# | 27# | 28# | 29# | 30# | 31# | — | — |
| Cross-sectional | 12.43 | 13.68 | 14.92 | 16.17 | 17.41 | 18.65 | 19.90 | 21.14 | 22.38 | — | — |
| Distance z (mm) | | | | | | | | | | | |
| Cross-sectional | 4.74 | 4.74 | 4.75 | 4.75 | 4.76 | 4.76 | 4.76 | 4.76 | 4.76 | — | — |
| Radius r (mm) | | | | | | | | | | | |

The above data were fitted using the mathematical model of the rotational generatrix of the contour shown in equation (1). The fitted values of the shape characteristic parameters r_{c}, rₙ, z₁, p₁, z₂, and p₂ are shown in Table 8.

TABLE 8
Fitting Results of Embodiment 3

| Parameter | r_{c} | rₙ | z₁ | p₁ | z₂ | p₂ |
|---|---|---|---|---|---|---|
| Fitted Value | 3.58954 | 4.76145 | 5.86727 | 5.05031 | 3.91829 | 1.89122 |
| Standard Error | 0.00488 | 0.00271 | 0.20075 | 0.37282 | 0.09469 | 0.07354 |

The coefficient of determination R² for the fitting is 0.99977, indicating that using the mathematical model shown in equation (1) can effectively describe the rotational generatrix of the contour of the specimen shape. Based on the fitting results, the obtained mathematical models describing the rotational generatrix of the contour of the specimen shape, the contour curved surface, and the tangent slope of the rotational generatrix of the contour in the plane formed by this line and the central axis are given by equations (10), (11), and (12), respectively.

$$r = 4.76145 + \frac{- 1.17191}{1 + {( \frac{z}{5.86727} )}^{5.05031} + {( \frac{z}{3.91829} )}^{1.89122}} \qquad ( 10 )$$

$$\sqrt{{x}^{2} + {y}^{2}} = 4.76145 + \frac{- 1.17191}{1 + {( \frac{z}{5.86727} )}^{5.05031} + {( \frac{z}{3.91829} )}^{1.89122}} \qquad ( 11 )$$

$${k}_{t} = \frac{7.78703 \times {10}^{- 4} \cdot {z}^{4.05031} + 0.16748 \cdot {z}^{0.89122}}{{[ 1 + {( \frac{z}{5.86727} )}^{5.05031} + {( \frac{z}{3.91829} )}^{1.89122} ]}^{2}} \qquad ( 12 )$$

Table 9 shows the values of the shape characteristic parameters of the specimen's necking deformation contour obtained according to the fitting process of Embodiment 3 and equations (12) and (10).

TABLE 9
Specimen Shape Characteristic Parameters in Embodiment 3

| | | | | Distance zᵢₚ | |
|---|---|---|---|---|---|
| | | | | between | |
| | | Maximum | | Cross-section | Cross- |
| | | Limit | | at | sectional |
| | | Value rₙ | | Inflection | Radius |
| | Minimum | of | Tangent | Point | rᵢₚ at |
| | Cross- | Cross- | Slope | Position and | Inflection |
| | sectional | sectional | kₜ^{ip} at | Minimum | Point |
| Characteristic | Radius r_{c} | Radius | Inflection | Cross-section | Position |
| Parameter | (mm) | (mm) | Point | (mm) | (mm) |
| Value | 3.58954 | 4.76145 | 0.19843 | 2.32713 | 3.91388 |

### Embodiment 4

Table 10 shows the measurement data of the cross-sectional radius r and the cross-sectional distance z at 31 points on the contour of the specimen during necking deformation in Embodiment 4.

TABLE 10
Contour Measurement Data in Embodiment 4

| | Measurement Point Number | | | | | | | | | | |
|---|---|---|---|---|---|---|---|---|---|---|---|
| | 1# | 2# | 3# | 4# | 5# | 6# | 7# | 8# | 9# | 10# | 11# |
| Cross-sectional | 0 | 0.50 | 0.99 | 1.49 | 1.98 | 2.48 | 2.98 | 3.47 | 3.97 | 4.46 | 4.96 |
| Distance z (mm) | | | | | | | | | | | |
| Cross-sectional | 3.35 | 3.36 | 3.41 | 3.49 | 3.61 | 3.67 | 3.84 | 3.97 | 4.08 | 4.20 | 4.31 |
| Radius r (mm) | | | | | | | | | | | |
| | Measurement Point Number | | | | | | | | | | |
| | 12# | 13# | 14# | 15# | 16# | 17# | 18# | 19# | 20# | 21# | 22# |
| Cross-sectional | 5.46 | 5.95 | 6.45 | 6.95 | 7.44 | 7.94 | 8.43 | 8.93 | 9.43 | 9.92 | 11.16 |
| Distance z (mm) | | | | | | | | | | | |
| Cross-sectional | 4.38 | 4.44 | 4.49 | 4.54 | 4.57 | 4.6 | 4.63 | 4.66 | 4.67 | 4.69 | 4.71 |
| Radius r (mm) | | | | | | | | | | | |
| | Measurement Point Number | | | | | | | | | | |
| | 23# | 24# | 25# | 26# | 27# | 28# | 29# | 30# | 31# | — | — |
| Cross-sectional | 12.40 | 13.64 | 14.88 | 16.12 | 17.36 | 18.60 | 19.84 | 21.08 | 22.32 | — | — |
| Distance z (mm) | | | | | | | | | | | |
| Cross-sectional | 4.73 | 4.73 | 4.74 | 4.75 | 4.76 | 4.76 | 4.76 | 4.76 | 4.76 | — | — |
| Radius r (mm) | | | | | | | | | | | |

The above data were fitted using the mathematical model of the rotational generatrix of the contour shown in equation (1). The fitted values of the shape characteristic parameters r_{c}, rₙ, z₁, p₁, z₂, and p₂ are shown in Table 11.

TABLE 11
Fitting Results of Embodiment 4

| Parameter | r_{c} | rₙ | z₁ | p₁ | z₂ | p₂ |
|---|---|---|---|---|---|---|
| Fitted Value | 3.34600 | 4.77053 | 4.42890 | 3.23009 | 7.17915 | 1.60469 |
| Standard Error | 0.00978 | 0.00598 | 0.52560 | 0.38041 | 4.81692 | 0.54412 |

The coefficient of determination R² for the fitting is 0.99955, indicating that using the mathematical model shown in equation (1) can effectively describe the rotational generatrix of the contour of the specimen shape. Based on the fitting results, the obtained mathematical models describing the rotational generatrix of the contour of the specimen shape, the contour curved surface, and the tangent slope of the rotational generatrix of the contour in the plane formed by this line and the central axis are given by equations (13), (14), and (15), respectively.

$$r = 4.77053 + \frac{- 1.42453}{1 + {( \frac{z}{4.4289} )}^{3.23009} + {( \frac{z}{7.17915} )}^{1.60469}} \qquad ( 13 )$$

$$\sqrt{{x}^{2} + {y}^{2}} = 4.77053 + \frac{- 1.42453}{1 + {( \frac{z}{4.4289} )}^{3.23009} + {( \frac{z}{7.17915} )}^{1.60469}} \qquad ( 14 )$$

$${k}_{t} = \frac{3.76091 \times 1 {0}^{2} \cdot {z}^{2.23009} + 0.09 \; 668 \cdot {z}^{0.60469}}{{[ 1 + {( \frac{z}{4.4289} )}^{3.23009} + {( \frac{z}{7.17915} )}^{1.60469} ]}^{2}} \qquad ( 15 )$$

Table 12 shows the values of the shape characteristic parameters of the specimen's necking deformation contour obtained according to the fitting process of Embodiment 4 and equations (15) and (13).

TABLE 12
Specimen Shape Characteristic Parameters in Embodiment 4

| | | | | Distance zᵢₚ | |
|---|---|---|---|---|---|
| | | | | between | |
| | | Maximum | | Cross-section | Cross- |
| | | Limit | | at | sectional |
| | | Value rₙ | | Inflection | Radius |
| | Minimum | of | Tangent | Point | rᵢₚ at |
| | Cross- | Cross- | Slope | Position and | Inflection |
| | sectional | sectional | kₜ^{ip} at | Minimum | Point |
| Characteristic | Radius r_{c} | Radius | Inflection | Cross-section | Position |
| Parameter | (mm) | (mm) | Point | (mm) | (mm) |
| Value | 3.346 | 4.77053 | 0.26622 | 3.02721 | 3.84713 |

### Embodiment 5

Table 13 shows the measurement data of the cross-sectional radius r and the cross-sectional distance z at 31 points on the contour of the specimen during necking deformation in Embodiment 5.

TABLE 13
Contour Measurement Data in Embodiment 5

| | Measurement Point Number | | | | | | | | | | |
|---|---|---|---|---|---|---|---|---|---|---|---|
| | 1# | 2# | 3# | 4# | 5# | 6# | 7# | 8# | 9# | 10# | 11# |
| Cross-sectional | 0 | 0.49 | 0.99 | 1.48 | 1.98 | 2.47 | 2.97 | 3.46 | 3.96 | 4.45 | 4.95 |
| Distance z (mm) | | | | | | | | | | | |
| Cross-sectional | 2.81 | 2.83 | 2.91 | 3.09 | 3.24 | 3.40 | 3.57 | 3.66 | 3.87 | 4.02 | 4.14 |
| Radius r (mm) | | | | | | | | | | | |
| | Measurement Point Number | | | | | | | | | | |
| | 12# | 13# | 14# | 15# | 16# | 17# | 18# | 19# | 20# | 21# | 22# |
| Cross-sectional | 5.44 | 5.94 | 6.43 | 6.93 | 7.42 | 7.92 | 8.41 | 8.91 | 9.40 | 9.90 | 11.13 |
| Distance z (mm) | | | | | | | | | | | |
| Cross-sectional | 4.24 | 4.33 | 4.39 | 4.45 | 4.51 | 4.54 | 4.58 | 4.61 | 4.63 | 4.65 | 4.68 |
| Radius r (mm) | | | | | | | | | | | |
| | Measurement Point Number | | | | | | | | | | |
| | 23# | 24# | 25# | 26# | 27# | 28# | 29# | 30# | 31# | — | — |
| Cross-sectional | 12.37 | 13.61 | 14.84 | 16.08 | 17.32 | 18.56 | 19.79 | 21.03 | 22.27 | — | — |
| Distance z (mm) | | | | | | | | | | | |
| Cross-sectional | 4.70 | 4.71 | 4.73 | 4.74 | 4.74 | 4.75 | 4.75 | 4.76 | 4.76 | — | — |
| Radius r (mm) | | | | | | | | | | | |

The above data were fitted using the mathematical model of the rotational generatrix of the contour shown in equation (1). The fitted values of the shape characteristic parameters r_{c}, rₙ, z₁, p₁, z₂, and p₂ are shown in Table 14.

TABLE 14
Fitting Results of Embodiment 5

| Parameter | r_{c} | rₙ | z₁ | p₁ | z₂ | p₂ |
|---|---|---|---|---|---|---|
| Fitted Value | 2.79552 | 4.75822 | 5.11829 | 3.90047 | 4.36624 | 1.72266 |
| Standard Error | 0.01425 | 0.00863 | 0.46598 | 0.51163 | 0.57935 | 0.20812 |

The coefficient of determination R for the fitting is 0.99941, indicating that using the mathematical model shown in equation (1) can effectively describe the rotational generatrix of the contour of the specimen shape. Based on the fitting results, the obtained mathematical models describing the rotational generatrix of the contour of the specimen shape, the contour curved surface, and the tangent slope of the rotational generatrix of the contour in the plane formed by this line and the central axis are given by equations (16), (17), and (18), respectively.

$$r = 4. 75822 + \frac{- 1.96270}{1 + {( \frac{z}{5.11829} )}^{3.90047} + {( \frac{z}{4.36624} )}^{1.72266}} \qquad ( 16 )$$

$$\sqrt{{x}^{2} + {y}^{2}} = 4.75822 + \frac{- 1.96270}{1 + {( \frac{z}{5.11829} )}^{3.90047} + {( \frac{z}{4.36624} )}^{1.72266}} \qquad ( 17 )$$

$${k}_{t} = \frac{1.31235 \times 1 {0}^{- 2} \cdot {z}^{2.90047} + 0.26 \; 691 \cdot {z}^{0.72266}}{{[ 1 + {( \frac{z}{5.11829} )}^{3.90047} + {( \frac{z}{4.36624} )}^{1.72266} ]}^{2}} \qquad ( 18 )$$

Table 15 shows the values of the shape characteristic parameters of the specimen's necking deformation contour obtained according to the fitting process of Embodiment 5 and equations (18) and (16).

TABLE 15
Specimen Shape Characteristic Parameters in Embodiment 5

| | | | | Distance zᵢₚ | |
|---|---|---|---|---|---|
| | | | | between | |
| | | Maximum | | Cross-section | Cross- |
| | | Limit | | at | sectional |
| | | Value rₙ | | Inflection | Radius |
| | Minimum | of | Tangent | Point | rᵢₚ at |
| | Cross- | Cross- | Slope | Position and | Inflection |
| | sectional | sectional | kₜ^{ip} at | Minimum | Point |
| Characteristic | Radius r_{c} | Radius | Inflection | Cross-section | Position |
| Parameter | (mm) | (mm) | Point | (mm) | (mm) |
| Value | 2.79552 | 4.75822 | 0.38853 | 2.62083 | 3.43971 |

Therefore, it can be seen that the mathematical model of the rotational generatrix of the specimen's necking deformation contour, the mathematical model of the contour curved surface, and the mathematical model of the tangent slope of the rotational generatrix of the contour in the plane formed by this line and the central axis, all constructed based on equation (1), can accurately describe the contour of a round bar specimen during uniaxial tensile necking deformation. The description is effective and highly accurate, providing a solid foundation for subsequent analysis of the stress field distribution in the necking region.

The above description is only for the purpose of illustrating the preferred embodiments of the present invention, and is not intended to limit the scope of the present invention. Any modifications, equivalents, improvements, and the like made without departing from the spirit and principle of the present invention shall fall in the protection scope of the present invention.

1. A method for analyzing a contour of a round bar specimen during uniaxial tensile necking deformation, wherein the method analyzes, by conducting a uniaxial tensile test on a round bar specimen, a shape of the specimen during a necking stage in the round bar tensile test, and comprises the following steps:
step S1, analyzing a shape of a contour line of the specimen during the necking stage;
step S2, setting hypothetical conditions, setting a rotational generatrix of a contour of the specimen at a necking bottom during the necking stage to be S-shaped, and establishing a mathematical model for the rotational generatrix as shown in equation (1)
$$r = {r}_{n} + \frac{{r}_{c} - {r}_{n}}{1 + {( \frac{z}{{z}_{1}} )}^{{p}_{1}} + {( \frac{z}{{z}_{2}} )}^{{p}_{2}}} \qquad ( 1 )$$
where r represents a cross-sectional radius perpendicular to a central axis at a position of any point on a contour curved surface of the specimen, z represents a distance between a cross-section at the any point and a minimum cross-section at the necking bottom, rₙ represents a maximum limit value of the cross-sectional radius perpendicular to the central axis, r_{c} represents a minimum cross-sectional radius at the necking bottom, and z₁, p₁, z₂, and p₂ represent undetermined shape characteristic parameters;
step S3, measuring test data of the specimen that has undergone necking deformation, wherein the test data comprises at least the cross-sectional radius r and the cross-sectional distance z, and there are a plurality of measurement points; substituting the measured test data into equation (1) for fitting to determine values of rₙ, r_{c}, and the shape characteristic parameters z₁, p₁, z₂, and p₂; and
step S4, substituting the values of rₙ, r_{c}, and the shape characteristic parameters z₁, p₁, z₂, and p₂ determined in step S3 into equation (1) to obtain a curve model of the rotational generatrix.

2. The method for analyzing a contour of a round bar specimen during uniaxial tensile necking deformation according to claim 1, wherein the hypothetical conditions in step S2 are as follows: during the necking stage of the uniaxial tensile test of the round bar specimen, a shape of the round bar specimen being a rotational body formed by rotating the rotational generatrix of the contour around the central axis; the specimen being symmetric with respect to the minimum cross-section at the necking bottom along a direction of the central axis; and a tangent line of a contour line at a position of the minimum cross-section is parallel to the central axis.

3. The method for analyzing a contour of a round bar specimen during uniaxial tensile necking deformation according to claim 1, wherein step S2 further comprises:
step S21, establishing a rectangular coordinate system with a center position of the minimum cross-section at the necking bottom, which is perpendicular to the central axis, as an origin, using the central axis as a z-axis of the coordinate system, and using any two mutually perpendicular radius lines intersecting at the center of the minimum cross-section at the necking bottom as an x-axis and a y-axis of the coordinate system; and
step S22, setting coordinates of any point on the contour curved surface of the specimen as (x, y, z), and according to the mathematical model of the rotational generatrix from step S2, establishing a curved surface model of the contour of the specimen as shown in equation (2)
$$\sqrt{{x}^{2} + {y}^{2}} = {r}_{n} + \frac{{r}_{c} - {r}_{n}}{1 + {( \frac{z}{{z}_{1}} )}^{{p}_{1}} + {( \frac{z}{{z}_{2}} )}^{{p}_{2}}} . \qquad ( 2 )$$

4. The method for analyzing a contour of a round bar specimen during uniaxial tensile necking deformation according to claim 3, wherein step S4 further comprises:
step S41, substituting the values of rₙ, r_{c}, and the shape characteristic parameters z₁, p₁, z₂, and p₂ determined in step S3 into equation (2) to obtain a curved surface model of the necking deformation contour.

5. The method for analyzing a contour of a round bar specimen during uniaxial tensile necking deformation according to claim 1, wherein step S2 further comprises:
step S21′, based on the mathematical model of the rotational generatrix in step S2, establishing a mathematical model of a tangent slope at any point on the rotational generatrix of the contour in a plane formed by the rotational generatrix and the central axis, as shown in equation (3)
$${k}_{t} = - ( {r}_{c} - {r}_{n} ) \cdot {[ 1 + {( \frac{z}{{z}_{1}} )}^{p 1} + {( \frac{z}{{z}_{2}} )}^{{p}_{2}} ]}^{- 2} \cdot [ \frac{{p}_{1}}{{z}_{1}^{p 1}} \cdot {z}^{{{p}_{1}}^{- 1}} + \frac{{p}_{2}}{{z}_{2}^{{p}_{2}}} \cdot {z}^{{{p}_{2}}^{- 1}} ] \qquad ( 3 )$$
where kₜ represents the tangent slope.

6. The method for analyzing a contour of a round bar specimen during uniaxial tensile necking deformation according to claim 5, wherein step S4 further comprises:
step S41′, substituting the values of rₙ, r_{c}, and the shape characteristic parameters z₁, p₁, z₂, and p₂ determined in step S3 into equation (3) to obtain the mathematical model of the tangent slope at any point on the rotational generatrix of the contour in the plane formed by the rotational generatrix and the central axis.

7. The method for analyzing a contour of a round bar specimen during uniaxial tensile necking deformation according to claim 6, wherein after step S41′, the following step is performed:
step S42′, acquiring a tangent slope according to the mathematical model established in step S41′, and determining a maximum value kₜ^{ip} of the tangent slope with a certain precision using a linear search method, the maximum value being a tangent slope at an inflection point position of the rotational generatrix of the contour,
wherein the linear search comprises: setting a series of z values, with a difference between two adjacent z values being a search step size Δz, where Δz represents search precision; calculating tangent slope kₜ corresponding to each z value according to equation (3); and identifying a maximum value of kₜ as kₜ^{ip}.

8. The method for analyzing a contour of a round bar specimen during uniaxial tensile necking deformation according to claim 7, wherein after step S42′, the following step is performed:
step S43′, substituting the z value corresponding to the maximum slope obtained in step S42′ into equation (1) to calculate and acquire a cross-sectional radius rᵢₚ perpendicular to the central axis at the inflection point position.

9. The method for analyzing a contour of a round bar specimen during uniaxial tensile necking deformation according to claim 1, wherein a number of the measurement points in step S3 is no less than 10.

10. The method for analyzing a contour of a round bar specimen during uniaxial tensile necking deformation according to claim 1, wherein a material of the round bar specimen is a metal material that undergoes necking deformation during the uniaxial tensile process of the round bar specimen.

11. The method for analyzing a contour of a round bar specimen during uniaxial tensile necking deformation according to claim 2, wherein a material of the round bar specimen is a metal material that undergoes necking deformation during the uniaxial tensile process of the round bar specimen.

12. The method for analyzing a contour of a round bar specimen during uniaxial tensile necking deformation according to claim 3, wherein a material of the round bar specimen is a metal material that undergoes necking deformation during the uniaxial tensile process of the round bar specimen.

13. The method for analyzing a contour of a round bar specimen during uniaxial tensile necking deformation according to claim 4, wherein a material of the round bar specimen is a metal material that undergoes necking deformation during the uniaxial tensile process of the round bar specimen.

14. The method for analyzing a contour of a round bar specimen during uniaxial tensile necking deformation according to claim 5, wherein a material of the round bar specimen is a metal material that undergoes necking deformation during the uniaxial tensile process of the round bar specimen.

15. The method for analyzing a contour of a round bar specimen during uniaxial tensile necking deformation according to claim 6, wherein a material of the round bar specimen is a metal material that undergoes necking deformation during the uniaxial tensile process of the round bar specimen.

16. The method for analyzing a contour of a round bar specimen during uniaxial tensile necking deformation according to claim 7, wherein a material of the round bar specimen is a metal material that undergoes necking deformation during the uniaxial tensile process of the round bar specimen.

17. The method for analyzing a contour of a round bar specimen during uniaxial tensile necking deformation according to claim 8, wherein a material of the round bar specimen is a metal material that undergoes necking deformation during the uniaxial tensile process of the round bar specimen.

18. The method for analyzing a contour of a round bar specimen during uniaxial tensile necking deformation according to claim 9, wherein a material of the round bar specimen is a metal material that undergoes necking deformation during the uniaxial tensile process of the round bar specimen.
